(19) United States
(12) Patent Application Publication
(10) Pub. No.: US 2011/0135756 A1
(43) Pub. Date: Jun. 9, 2011

(54) COMPOSITIONS AND METHODS FOR PROTECTING SENSORY HAIR CELLS

(75) Inventors: Kelly N. Owens, Seattle, WA (US); Anna L. Corke, Seattle, WA (US); Henry C. Ou, Seattle, WA (US); Edwin W. Rubel, Seattle, WA (US); David W. Raible, Seattle, WA (US); Julian A. Simon, Seattle, WA (US)

(73) Assignees: University of Washington, Seattle, WA (US); Fred Hutchinson Cancer Research Center, Seattle, WA (US)

(21) Appl. No.: 12/963,396

(22) Filed: Dec. 8, 2010

Related U.S. Application Data
(60) Provisional application No. 61/267,789, filed on Dec. 8, 2009.

(51) Int. Cl. A61K 33/24; A61K 31/522; A61K 31/122; A61K 31/4965; A61K 31/546; A61K 31/4706; A61K 31/138; A61K 31/438; A61K 31/7036; A61P 17/00; A61P 27/16

(52) U.S. Cl. 424/649; 514/263.34; 514/682; 514/255.06; 514/202; 514/313; 514/651; 514/278; 514/252.16; 514/39

(57) ABSTRACT

The invention provides compounds, compositions and methods that can be used for the attenuation of damage to sensory hair cells and symptoms thereof. More particularly, the invention identifies drugs that can be used to protect sensory hair cells from ototoxic medications, noise-induced damage and age-related loss.

This application claims the benefit of U.S. provisional patent application No. 61/267,789, filed Dec. 8, 2009, the entire contents of which are incorporated herein by reference.

### STATEMENT REGARDING FEDERALLY SPONSORED RESEARCH

This invention was made with government support under grant numbers R01 DC05987 and P30 DC04661 awarded by the National Institutes of Health. The government has certain rights in the invention.

### TECHNICAL FIELD OF THE INVENTION

The invention relates to compounds, compositions and methods that can be used for the attenuation of damage to sensory hair cells and symptoms thereof. More particularly, the invention identifies drugs that can be used to protect sensory hair cells from ototoxic medications, noise-induced damage and age-related loss.

### BACKGROUND OF THE INVENTION

Hair cells of the inner ear are critical to hearing and vestibular function. In mammals, the loss of sensory hair cells is permanent, as there is no significant capacity for regeneration of these cells. Drugs such as aminoglycoside antibiotics and many anti-neoplastic drugs are often used despite unfortunate side effects. One such side effect is hearing loss due to death of the sensory hair cells of the inner ear. Aminoglycosides are clinically used drugs that cause dose-dependent sensorineural hearing loss (Smith et al., New Engl J Med. (1977) 296:349-53) and are known to kill hair cells in the mammalian inner ear (Theopold, Acta Otolaryngol (1977) 84:57-64). In the U.S. over 2,000,000 people receive treatment with aminoglycosides per year. The clinical efficacy of these drugs in treating resistant bacterial infections and their low cost globally account for their continued use and need. Cisplatin, a chemotherapeutic agent, is also used for its benefit to life despite its toxic effects on the hair cells of the inner ear. High frequency hearing loss (>8 kHZ) has been reported to be as high as 90% in children undergoing cisplatin therapy (Allen, et al., Otolaryngol Head Neck Surg (1998) 118:584-588). The incidence of vestibulotoxic effects of such drugs on patient populations has been less well studied. Estimates range between 3% and 6% with continued reports in the literature of patients with aminoglycoside induced vestibulotoxicity (Dhanireddy et al., Arch Otolarngol Head Neck Surg (2005) 131:46-48). Other clinically important and commonly used drugs also have documented ototoxic effects, including loop diuretics (Greenberg, Am J Med Sci. (2000) 319:10-24) and antimalarial quinines (Claessen, et al., Trop Med Int Health, (1998) 3:482-9) salicylates (Matz, Ann Otol Rhinol Laryngol Suppl (1990) 148:39-41).

Research in the past few decades has uncovered some of the key intracellular events that can cause hair cell death. Several candidate protectants have been evaluated such as anti-oxidants, caspase inhibitors, and jun kinase inhibitors (Kopke R D, et al. Am J Otol 1997, 18:559-571; Liu W, et al. Neuroreport 1998, 9:2609-2614; Yamasoba T. et al. Brain Res 1999, 815:317-325: Matsui J I, et al. J Neurosci 2002, 22:1218-1227; Sugahara K, et al. Hear Res 2006, 221:128-135.) Although a few of these candidate otoprotectants have progressed to human trials (Sha S H, et al. N Engl J Med 2006, 354:1856-1857; Campbell K C, et al. Hear Res 2007, 226:92-103) as yet, no definitive protection has emerged for clinical use. Further, different cell death pathways may be triggered in response to different forms of damage, and many protective molecules offer incomplete hair cell protection, hinting that polypharmacy approaches may offer the greatest benefit. Given the difficulty of assessing many putative hair cell protectants for efficacy against multiple ototoxins, the field has proceeded slowly.

There remains a need to identify compounds and methods for protecting sensory hair cells from ototoxic damage and death. There remains an ongoing need to identify protectants effective against the many different ototoxic medications across the range of doses in clinical use. In addition, there remains a need to identify protectants against other insults to sensory hair cells, including noise and aging.

### SUMMARY OF THE INVENTION

The invention is based on the discovery of protective drugs that ameliorate sensory hair cell loss. Such loss can be the result of exposure to ototoxic medications, noise damage, and/or aging. In one embodiment, the invention provides drugs that have been demonstrated to protect sensory hair cells against the toxic effects of aminoglycoside antibiotics and/or other ototoxic medications.

All of the drugs listed below are FDA-approved for other uses, but not previously used to prevent drug-induced hearing loss. The following drugs provide new methods of protecting against hearing loss and other symptoms of sensory hair cell damage:

Drug Name (CAS #)

Aminophylline (317-34-0)

Atovaquone (95233-18-4)

Benzamil (2898-76-2)

Cefepime (88040-23-7)

Chloroquine phosphate (50-63-5)

Fluoxetine HCl (56296-78-7)

Fluperlapine (67121-76-0)

Fluspirilene (1841-19)

Loperamide (34552-83-5)

Methiothepin maleate (19728-88-2)

Paroxetine HCl (110429-49-8)

Phenoxybenzamine HCl (63-92-3)

Ractopamine (97825-25-7)

Raloxifene HCl (82640-04-8)

Sildenafil (139755-83-2)

Tamoxifen citrate (54965-24-1)

Ticlopidine HCl (53885-35-1)

Trequinsin (79855-88-2)

Trifluperidol 2HCl (749-13-3)

Toremifene (89778-26-7)

The following protective drugs have been confirmed to achieve the protective effect without diminishing the antibiotic efficacy of the ototoxic aminoglycoside: loperamide, ractopamine, raloxifene, paroxetine, phenoxybenzamine, chloroquine, methiothepin, fluoxetine, fluspirilene, tamoxifen, and toremifene. In addition, benzamil does not alter the ability of the antibiotic to inhibit bacterial growth, although it does alter the dose needed to kill bacteria. Each of these drugs has been shown to protect sensory hair cells from neomycin. In addition, benzamil, loperamide, ractopamine, raloxifene, paroxetine, phenoxybenzamine, chloroquine, and methiothepin were all shown to protect against gentamicin. Benzamil, loperamide, ractopamine were shown to protect against kanamycin. All of these protective drugs were also shown to be protective across a broad range of aminoglycoside doses tested, up to 400 μm, the highest tested dose. Benzamil and paroxetine were additionally protective against cisplatin, also at a broad range of doses tested, up to 100 μM cisplatin, the highest tested dose. Benzamil has been confirmed as not altering the ability of cisplatin to inhibit growth of a human cancer cell line. Paroxetine alters inhibition of growth of some cultured lung cancer cells at some doses and not at others, based on in vitro testing. Additional protective drugs can be confirmed as protective without diminishing efficacy of ototoxic medication through use of the assays described in the Examples below.

Certain protective drugs provided by the invention, namely methiothepin and phenoxybenzamine, were more effective when the sensory hair cells were pre-treated 15 minutes (phenoxybenzamine) or 60 minutes (methiothepin) prior to aminoglycoside exposure. All of the other agents tested were effective when co-administered with the aminoglycoside.

The invention additionally provides pharmaceutical compositions comprising one or more protective drugs of the invention, optionally in combination with at least one ototoxic medication. The composition can optionally comprise a pharmaceutically acceptable carrier and/or excipient.

Further confirmation of the applicability of these protective compounds to clinical conditions is provided by assays performed in mammals. Example 3 below describes use of a rat model of aminoglycoside-induced hearing loss that closely mimics the pattern of hearing loss observed in human patients treated with aminoglycosides. In this example, a drug shown to protect sensory hair cells in zebrafish against kanamycin exposure was also shown to protect against hearing loss in rats treated with kanamycin.

The protective drugs of the invention can be administered locally or systemically. The administration can be oral, intraperitoneal, intramuscular, intra-aural, transtympanic or intravenous. The protective drug can be co-administered with an ototoxic medication, or administered separately, whether at the same time or as a pre- or post-treatment.

The invention thus provides a method of attenuating sensory hair cell death in a subject. The method comprises administering to the subject a sufficient amount of a protective drug selected from the group consisting of: aminophylline (317-34-0), atovaquone (95233-18-4), benzamil (2898-76-2), cefepime (88040-23-7), chloroquine phosphate (50-63-5), fluoxetine HCl (56296-78-7), fluperlapine (67121-76-0), fluspirilene (1841-19), loperamide (34552-83-5), methiothepin maleate (19728-88-2), paroxetine HCl (110429-49-8), phenoxybenzamine HCl (63-92-3), ractopamine (97825-25-7), raloxifene HCl (82640-04-8), sildenafil (139755-83-2), tamoxifen citrate (54965-24-1), ticlopidine HCl (53885-35-1), trequinsin (79855-88-2), trifluperidol 2HCl (749-13-3), toremifene (89778-26-7), quinine, cinchonine, cinchonidine, mefloquine, aminacrine, tacrine, amsacrine, and amodiaquine.

Also provided is a method of reducing ototoxic effects of ototoxic medication in a subject. Additional methods provided by the invention include: a method of reducing hearing loss in a subject treated with ototoxic medication, and a method of attenuating or blocking aminoglycoside entry into cells in a subject. The methods comprise administering to the subject a sufficient amount of a protective drug selected from the drugs listed above.

In some embodiments, the protective drug may be administered at or near the same time (e.g., within about 5-10 minutes) as the administration of the ototoxic medication. In other embodiments, the protective drug is significantly more effective when administered prior to the administration of the ototoxic medication. Typically, the prior administration is at least about 15 to about 60 minutes prior to the administration of ototoxic medication. Pre-treatment can be 30, 45, 60 or 90 minutes, or up to about 24 hours before ototoxic medication is administered. In some embodiments, the protective drug is administered within a short interval after administration of the ototoxic medication. A typical short interval is about 5 to about 60 minutes. In some embodiments, the short interval is up to 24-72 hours. For medications in which the progression of hearing loss is typically delayed (e.g. as has been observed with cisplatin), the protective drug may still be effective after a much longer interval (e.g., months or years). The examples provided below offer extensive guidance in how one can optimize and extend the options for timing and dosage for a given combination of protective drug and ototoxic exposure.

### BRIEF DESCRIPTION OF THE FIGURE

FIG. 1 is a bar graph depicting results of cinchonine pretreatment, which protects against neomycin-induced hair cell death. Five day post-fertilization zebrafish were pretreated with 0, 10, 50, 100 or 200 μM cinchonine prior to treatment with 200 μM neomycin. With increasing doses of cinchonine, there was increasing hair cell survival. The negative control represents fish not exposed to neomycin. Data bars are mean hair cell survival from 8 to 10 fish. Error bars represent standard deviation from the mean.

### DETAILED DESCRIPTION OF THE INVENTION

The invention described herein is based on the discovery of protective drugs that ameliorate sensory hair cell loss. To discover compounds that counteract drug-induced hair cell toxicity, we screened the BIOMOL 640 library (Enzo Life Sciences) for drugs that protect hair cells of the zebrafish lateral line from the toxic effects of several aminoglycosides (neomycin, gentamicin, kanamycin) or the platinum compound, cisplatin. Additional screening was performed with the NINDS Custom Collection II, now called the U.S. Drug Collection by the supplier (Microsource Discovery Systems. Inc., Gaylordsville, Conn.) The hair cells of the zebrafish lateral line have emerged as a valuable in vivo model system to screen for genetic and chemical modulators of hair cell death (described in U.S. patent application Ser. No. 12/014,470, filed Jan. 15, 2008, and published as US 2009 0023751-A1 on Jan. 22, 2009).

Of the 640 drugs screened, 20 drugs were identified that confer protection against at least one aminoglycoside and/or cisplatin, including loperamide, which protects against all four toxins. This screen revealed drugs that had been identified as protective in previous studies: phenoxybenzamine, which was found in a screen of a different drug library (Ou et al. 2009 JARO 10(2)191-203) and benzamil, which is a derivative of amiloride, a known protectant (Owens et al. 2009 Hear Res 253(1-2)32-41).

Further testing of 12 of these drugs revealed dose-dependency of protection when the dose of the putative protective compound was varied. Using fluorescently-conjugated gentamicin, we identified compounds that block gentamicin entry into hair cells, and others that do not. Drugs that blocked gentamicin entry also showed robust protection against other aminoglycosides. In a particular example, ractopamine protected hair cells against neomycin, gentamicin and kanamycin at a wide range of doses. The invention provides a number of drugs that protect sensory hair cells across a broad range of ototoxic medication doses, making them particularly attractive for practical clinical application.

Several of the protective compounds fall into functional or structural categories, suggesting common mechanisms of protection. Fluoxetine and paroxetine are both selective serotonin reuptake inhibitors, while tamoxifen and raloxifene are both estrogen receptor modulators. Differences in the protective profiles of drugs within each class suggest that the structural and functional similarities may not fully explain their protective effects.

### DEFINITIONS

All scientific and technical terms used in this application have meanings commonly used in the art unless otherwise specified. As used in this application, the following words or phrases have the meanings specified.

Representative ototoxic effects include: hearing loss, sensory hair cell death, tinnitus vertigo and dizziness. In addition, many ototoxic medications also cause kidney failure or damage. The protective drugs of the invention may also be used to protect kidney cells.

Antibiotic medications include aminoglycosides, such as neomycin, gentamicin, kanamycin, tobramycin, amikacin.

Anti-neoplastic medications include the platinum compound, cisplatin, and its derivatives such as carboplatin.

As used herein, a drug is “protective” against sensory hair cell death if it attenuates hair cell loss and/or symptoms of hair cell damage relative to the ototoxic effects observed in the absence of protective drug treatment.

As used herein, “pharmaceutically acceptable salt” refers to a salt that retains the desired biological activity of the parent compound and does not impart any undesired toxicological effects. Examples of such salts include, but are not limited to, (a) acid addition salts formed with inorganic acids, for example hydrochloric acid, hydrobromic acid, sulfuric acid, phosphoric acid, nitric acid and the like; and salts formed with organic acids such as, for example, acetic acid, oxalic acid, tartaric acid, succinic acid, maleic acid, furmaric acid, gluconic acid, citric acid, malic acid, ascorbic acid, benzoic acid, tannic acid, pamoic acid, alginic acid, polyglutamic acid, naphthalenesulfonic acids, naphthalenedisulfonic acids, polygalacturonic acid; (b) salts with polyvalent metal cations such as zinc, calcium, bismuth, barium, magnesium, aluminum, copper, cobalt, nickel, cadmium, and the like; or (c) salts formed with an organic cation formed from N,N′-dibenzylethylenediamine or ethylenediamine; or (d) combinations of (a) and (b) or (c), e.g. a zinc tannate salt; and the like. The preferred acid addition salts are the trifluoroacetate salt and the acetate salt.

As used herein, “pharmaceutically acceptable carrier” or “excipient” includes any material which, when combined with an active ingredient, allows the ingredient to retain biological activity and is non-reactive with the subject's immune system. Examples include, but are not limited to, any of the standard pharmaceutical carriers such as a phosphate buffered saline solution, water, emulsions such as oil/water emulsion, and various types of wetting agents. Preferred diluents for aerosol or parenteral administration are phosphate buffered saline or normal (0.9%) saline.

Compositions comprising such carriers are formulated by well known conventional methods (see, for example, Remington's Pharmaceutical Sciences, 18th edition, A. Gennaro, ed., Mack Publishing Co., Easton, Pa., 1990).

As used herein, “a” or “an” means at least one, unless clearly indicated otherwise.

As used herein, to “prevent” or “protect against” a condition or event means to hinder, reduce or delay the onset or progression of the condition or event.

### Compositions

The invention provides compositions that are useful for preventing or attenuating sensory hair cell damage and symptoms thereof. The compositions can be used in the methods described herein. In one embodiment, the composition is a pharmaceutical composition. The composition can comprise a sufficient or prophylactically effective amount of one or more protective drugs of the invention. An effective amount is an amount sufficient to reduce the symptoms of hair cell damage relative to the ototoxic effects observed in comparable subject in the absence of protective drug treatment.

A pharmaceutical composition may contain one or more protective drugs of the invention and, optionally, an ototoxic medication to be administered simultaneously with the protectant. Alternatively, the protective drug(s) may be administered as a separate composition, either at the same time as the ototoxic medication, or as a pre- or post-treatment.

The composition can optionally include a carrier, such as a pharmaceutically acceptable carrier. Pharmaceutically acceptable carriers are determined in part by the particular composition being administered, as well as by the mode of administration. Accordingly, there is a wide variety of suitable formulations of pharmaceutical compositions of the present invention. Formulations suitable for parenteral administration, such as, for example, by intravenous, intramuscular, intradermal, intraperitoneal, and subcutaneous routes, and carriers include aqueous isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, preservatives and emulsions. For oral administration, any of the above carriers or a solid carrier, such as mannitol, lactose, starch, magnesium stearate, sodium saccharine, talcum, cellulose, glucose, sucrose, and magnesium carbonate, may be employed. In some embodiments, a slow-release formulation is desirable for local or for systemic administration. One example of a slow-release formulation is a gel, which could be used for local administration. Local administration includes delivery to the inner ear.

Compositions are typically administered in vivo via parenteral (e.g. intravenous, subcutaneous, and intramuscular) or other traditional direct routes, or directly into a specific tissue. For example, local administration can be achieved by transtympanic injection. Suitable methods of administering cells in the context of the present invention to a patient are available, and, although more than one route can be used to administer a particular cell composition, a particular route can often provide a more immediate and more effective reaction than another route.

The dose will be determined by the activity of the composition produced and the condition of the patient, as well as the body weight or surface areas of the patient to be treated. The size of the dose also will be determined by the existence, nature, and extent of any adverse side effects that accompany the administration of a particular composition in a particular patient. Preferably, a dosage is selected such that a single dose will suffice or, alternatively, several doses are administered over the course of several months.

### Methods of Protection

The invention provides a method of attenuating sensory hair cell death in a subject. Also provided is a method of reducing ototoxic effects of antibiotic, anti-neoplastic or other chemotherapeutic medication in a subject. Additional methods provided by the invention include: a method of reducing hearing loss in a subject treated with antibiotic or anti-neoplastic medication or other ototoxin, and a method of attenuating or blocking aminoglycoside entry into cells in a subject.

The method comprises administering to the subject a sufficient amount of a protective drug selected from the group consisting of: aminophylline (317-34-0), atovaquone (95233-18-4), benzamil (2898-76-2), cefepime (88040-23-7), chloroquine phosphate (50-63-5), fluoxetine HCl (56296-78-7), fluperlapine (67121-76-0), fluspirilene (1841-19), loperamide (34552-83-5), methiothepin maleate (19728-88-2), paroxetine HCl (110429-49-8), phenoxybenzamine HCl (63-92-3), ractopamine (97825-25-7), raloxifene HCl (82640-04-8), sildenafil (139755-83-2), tamoxifen citrate (54965-24-1), ticlopidine HCl (53885-35-1), trequinsin (79855-88-2), trifluperidol 2HCl (749-13-3), toremifene (89778-26-7), quinine, cinchonine, cinchonidine, mefloquine, aminacrine, tacrine, amsacrine, and amodiaquine.

The invention provides a method of attenuating sensory hair cell damage in a subject treated with aminoglycoside antibiotics and other therapeutic agents. The method comprises administering to the subject one or more protective drugs of the invention. In one embodiment, the subject is treated with up to 2 mg/kg, or up to 5 mg/kg or higher, of antibiotic. Partial protection, which may occur at some higher doses of antibiotic would continue to be of clinical benefit.

In some embodiments, the protective drug may be administered at or near the same time (e.g., within about 5-10 minutes) as the administration of the ototoxic medication. In other embodiments, the protective drug is significantly more effective when administered prior to the administration of the ototoxic medication. Typically, the prior administration is at least about 15 to about 60 minutes prior to the administration of ototoxic medication. Pre-treatment can be 30, 45, 60 or 90 minutes, 24, 36, 72 hours, or within the week before ototoxic medication is administered. In some embodiments, the protective drug is administered within a short interval after administration of the ototoxic medication. A typical short interval is about 5 to about 60 minutes. In some embodiments, the short interval is up to 24-72 hours, or up to a week. It is understood that, although these time intervals for pre- and post-treatment are most typical, the timing that will be effective for a particular patient, a particular ototoxic insult, and/or a particular treatment environment can vary beyond these parameters. For medications in which the progression of hearing loss is typically delayed (e.g. as has been observed with cisplatin), the protective drug may still be effective after a much longer interval (e.g., months or years). The examples provided below offer extensive guidance in how one can optimize and extend the options for timing and dosage for a given combination of protective drug and ototoxic exposure.

The protective drugs of the invention can be administered locally or systemically. The administration can be oral, intraperitoneal, intramuscular, intra-aural, transtympanic or intravenous. The protective drug can be delivered to the ear using a variety of methods, including direct injection or surgically implanting a means for slow release of the protectant over time, such as embedded in a hydrogel placed in or near the ear, or in a pump.

### EXAMPLES

The following examples are presented to illustrate the present invention and to assist one of ordinary skill in making and using the same. The examples are not intended in any way to otherwise limit the scope of the invention.

### Example 1

### Screening for Protective FDA-Approved Drugs

Animal care. Wildtype Larval zebrafish (Danio rerio) were produced via group matings of adult fish. Larvae were housed at 28.5 C and maintained at a density of 50 fish per 10 cm diameter petri dish in embryo media (994 μM MgSO₄, 150 μM KH₂PO₄, 42 μM Na₂HPO₄, 986 μM CaCl₂, 503 μM KCl, 14.9 mM NaCl, and 714 μM NaHCO₃, with the pH adjusted to 7.2.). Beginning at 4 days post-fertilization (dpf), fish were fed live paramecia or rotifers daily. Experiments were performed using 5 or 6 dpf larvae. The University of Washington Animal Care and Use Committee approved of the animal procedures described here.

Drug Library. BIOMOL's FDA Approved Drug Library (Enzo Life Sciences Inc., Plymouth Meeting, Pa., USA (formerly BIOMOL International, L.P.)) was used to screen zebrafish larvae for compounds that protect against toxin-induced hair cell death. The library consists of 640 drugs dissolved at 2 mg/ml in dimethyl sulfoxide (DMSO). The drugs were aliquoted into eight 96-well plates with 80 drugs per plate and stored at 4 C during initial screening and re-testing.

Screening. Larvae were prelabeled with 2 μM YO-PRO1 (Invitrogen, Carlsbad, Calif. USA; Y3603) in embryo medium for 30 min and then rinsed three times. YO-PRO1 is a cyanine monomer fluorescent vital dye that labels hair cell DNA (Santos et al. 2006). After prelabeling, larvae were transferred to Nunc 96-well optical bottom plates (Thermo Fisher Scientific), one fish with 147 μL of embryo medium in each well. Library compounds were diluted 1:10 in embryo medium and then 3 μL of the diluted mixture were added to 96 well plate containing larvae (one drug per well) for a final drug concentration of 4 μg/ml of library compound and final DMSO concentration of 0.2% in each well. Larvae were incubated for 1 hr with library compounds. Then one of the following hair cell toxins was added and fish were incubated in library compound and hair cell toxin together for a duration sufficient to kill most hair cells.

Drug, Concentration, Incubation Time

### Neomycin, 200 μM, 1 hr

### Gentamicin, 50 μM, 6 hrs

### Kanamycin, 400 μM, 24 hrs

### Cisplatin, 50 μM, 24 hrs

Concentrations and incubation times were chosen based on previously determined dose-response in order to achieve maximal hair cell death at the lowest concentration of toxin (Ou et al. 2007; Owens et al. 2009). Eight fish in each plate served as mock controls and received no treatment. Eight fish were treated with hair cell toxin but not library drugs to control for toxin potency. After incubation in library drug and hair cell toxin, larvae were anesthetized with 0.001% MS222 (3-aminobenzoic acid ethyl ester methanesulfonate; Sigma) and immediately viewed using fluorescence microscopy on an automated stage (Marianas imaging system, Intelligent Imaging Innovations) using a Zeiss Axiovert 200M inverted microscope (Carl Zeiss). Fish were scored on a scale from 0 to 2 with 2 corresponding to mostly healthy hair cells and a 0 corresponding to mostly dead hair cells. Additionally heartbeats were monitored to determine whether fish survived the protocol and scores corresponding to dead larvae were discarded. Drugs from those wells were retested in triplicate to verify ototoxicity to fish. Drugs that scored a 2 were retested on five larvae with the above protocol, and those that scored a mean of 1.5 or greater in retesting were considered “hits”. Screening one plate took approximately 40 min.

Dose response testing. Drug screen “hits” were tested to determine minimum concentrations of drug and maximum concentrations of toxin that confer protection against hair cell death. All “hits” were tested against all four hair cell toxins regardless of whether a given “hit” was positive with a specific toxin in the screen. 5 or 6 dpf larvae were transferred to 6-well Corning Netwell baskets and placed in 6-well plates (Fisher Scientific) in EM with approximately 10 fish per basket. This allowed for easy transfer between treatment media. Drug and toxins were made up in 7 ml of EM. Larvae were pretreated for one hour in a dose of protective drug followed by cotreatment in protective drug and hair cell toxin. Incubation times in toxins were the same as those used for the 96 well drug screen format (see above) with the exception that gentamicin was also tested with a 1 hr treatment in addition to the 6 hr treatment point for drugs that did not show protection with the 6 hr gentamicin exposure. After drug and toxin treatments were complete, the larvae were rinsed 4× in EM and treated with 0.005% of DASPEI (2-(4-(dimethylamino)styryl)-N-ethylpyridinium iodide; Sigma, St. Louis Mo.) in EM for 15 min to label neuromasts. Then larvae were rinsed 4× and anesthetized with MS222. Larvae were transferred to glass depression slides and viewed on a Leica epifluorescent microscope with a DASPEI filter (Chroma Technologies, Brattleboro Vt.). Neuromasts were scored as previously described (Owens et al. 2009). To find minimum protective concentrations, doses of protective drug tested were 0, 0.5, 1, 5, 10, 50, 100 uM and toxin dose was held at the concentrations used for screening (see above). For those doses of putative protectants that killed the larvae, further doses were tested to determine if intermediate doses were optimal. For dose response curves finding maximum toxin dose at which protection occurs, dose of protective drug was held at previously determined optimal dose and toxin dose was varied as follows:

Neomycin: 0, 25, 50, 100, 200, 400

Gentamicin: 0, 25, 50, 100, 200, 400

Kanamycin: 0, 25, 50, 100, 200, 400

Cisplatin: 0, 5, 10, 25, 50, 100

Pretreatment experiments. To test whether a 1 hr pretreatment in protective drug is necessary for the protective effects, larvae were pretreated in protective drug for 1 hr, 15 min, or not at all followed by 1 hr cotreatment in protective drug and 200 uM neomycin using the dose response protocol described above. Controls were mock treated or treated with neomycin only.

Gentamicin-conjugated Texas Red Imaging. Gentamicin-conjugated to Texas Red (GTTR) was prepared following Steyger et al. 2003. To determine whether gentamicin is able to enter hair cells in the presence of protective drug, larvae were pretreated with protective drug at optimal dose for time determined in pretreatment experiments (above) followed by cotreatment with 50 uM GTTR for 3 min. Fish were anesthetized with MS222 and transferred to double wholemount slides for imaging using fluorescence microscopy on an automated stage (Marianas imaging system, Intelligent Imaging Innovations) with a Zeiss Axiovert 200M inverted microscope (Carl Zeiss). Each image contained a z-stack encompassing 2-3 neuromasts as determined by viewing under brightfield illumination. Each fish was imaged once and 5 fish were imaged for each treatment group. Control fish were treated with EM only (mock-treated) or with 3 min of 50 uM GTTR only.

GTTR Image analysis. Image analysis was done using Slidebook 5 (Intelligent Imaging Innovations, Denver Colo.) and Excel 2003 (Microsoft, Redmond, Wash.). To semi-quantitatively measure the amount of GTTR uptake that occurred in each treatment group, images had background image subtracted and were flat-field corrected. Then 2-3 neuromasts per image were traced and converted to a mask field “Mask 1”. The trace borders were then moved to a nearby background region of the image on the fish that did not contain neuromasts and a second mask was created “Mask 2.” The mean and standard deviation (SD) of the intensity of Mask 2 was used to create a thresholded segment mask with minimum intensity of mean+2SD of Mask 2. Then Boolean addition was used to create the neuromast mask (NM) by taking “Mask1” AND thresholded segment and the background mask (BG) by taking “Mask2” AND thresholded segment. Mean intensity, standard deviation of intensity, sum intensity and volume in voxels were calculated for NM and BG. To create an index of intensity, NM/BG was calculated for each fish and values for mock-treated fish were subtracted and multiplied by 100 to obtain “% intensity above background”.

TABLE 1
Screen results

| | | | Drug | Pre- | |
|---|---|---|---|---|---|
| | | | dose | treat | |
| Protective Drug | CAS # | Toxins | (μM) | (min) | Uptake |
| Benzamil | 2898-76-2 | N, G6, | 50 | 0 | attenuate |
| | | K, C | | | |
| Chloroquine | 50-63-5 | N, G6 | 50 | 0 | not done |
| diphosphate | | | | | |
| Fluoxetine HCl | 56296-78-7 | N, G1 | 50 | 0 | no block |
| Fluspirilene | 1841-19-6 | N, G1 | 10 | 0 | no block |
| Loperamide | 34552-83-5 | N, G6, | 10 | 0 | no block |
| | | K | | | |
| Methiothepin | 19728-88-2 | N, G6 | 10 | 60 | no block |
| maleate | | | | | |
| Paroxetine HCl | 110429-49-8 | N, G6, C | 10 | 0 | attenuate |
| Phenoxybenzamine | 63-92-3 | N, G6 | 50 | 15 | attenuate |
| HCl | | | | | |
| Ractopamine | 97825-25-7 | N, G6, | 50 | 0 | block |
| | | K | | | |
| Raloxifene HCl | 82640-04-8 | N, G6 | 10 | 0 | block |
| Tamoxifen citrate | 54965-24-1 | N, G1 | 10 | 0 | no block |
| Toremifene citrate | 89778-27-8 | N, G1 | 10 | not | not done |
| | | | | done | |

Drugs from an FDA-approved drug library that protect hair cells from toxin-induced cell death.
The “toxins” column shows which toxins a given drug protects against.
N = neomycin,
G1 = 1 hr gentamicin exposure,
G6 = 6 hr gentamicin exposure,
K = kanamycin,
C = cisplatin.
“Drug dose” is the optimal dose in μM that confers protection against toxins.
“Toxin dose” is the maximal toxin dose at which protective drug is effective.
“Pretreat” is the necessary pretreatment time before toxin exposure for effective protection.
“Uptake” lists whether gentamicin is able to enter cells in the presence of protective drug.
Block = no toxin entry;
attenuate = slowed, delayed or reduced uptake;
no block = gentamicin entry comparable to controls.

TABLE 2
Targets of Protective Drugs

| Protective Drug | FDA target |
|---|---|
| Benzamil | Na/Ca channel blocker |
| Chloroquine diphosphate | Antimalarial |
| Fluoxetine HCl | SSRI |
| Fluspirilene | Dopamine antagonist |
| Loperamide | μ-opioid receptor agonist |
| Methiothepin maleate | Serotonin and dopamine agonist |
| Paroxetine HCl | SSRI |
| Phenoxybenzamine HCl | antagonist at alpha adrenoceptor |
| Ractopamine | agonist at beta adrenoceptor |
| Raloxifene HCl | SERM |
| Tamoxifen citrate | SERM |
| Toremifene citrate | SERM |

The drugs from an FDA-approved drug library that protect hair cells from toxin-induced cell death as listed in Table 1 above are listed here with their corresponding “FDA targets”.
“FDA target” is the accepted target of the drug in the literature, which may or may not describe the mode of action when protecting hair cells.
SSRI = selective serotonin reuptake inhibitor
SERM = selective estrogen receptor modulator

### Example 2

### Quinoline Ring Derivatives Protect Against Aminoglycoside-Induced Hair Cell Death

This Example describes nine drugs with quinoline ring structures that prevent aminoglycoside-induced hair cell death. This finding suggests that quinoline ring structures can be used as a foundation for developing drugs that can be used to protect against inner ear damage. The nine drugs have a common quinoline ring structure (two linked six-member aromatic rings with one nitrogen):

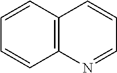

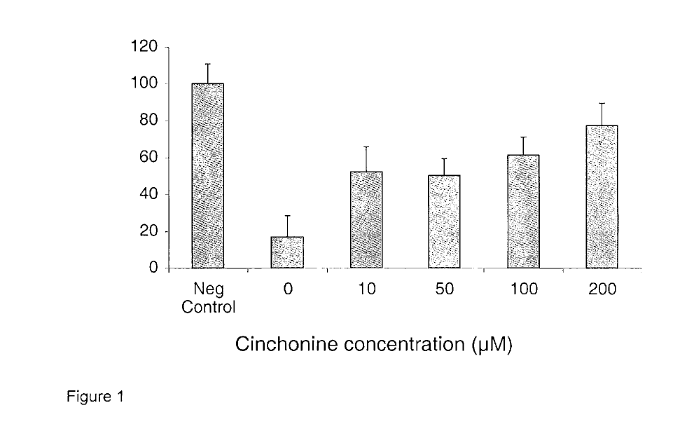

An example of the protection afforded by one of the nine drugs, cinchonine, is shown in FIG. 1. With pretreatment with cinchonine, there is increasing hair cell survival and protection against treatment with 200 μM neomycin.

FIG. 1 shows that cinchonine pretreatment protects against neomycin-induced hair cell death. Five day post-fertilization zebrafish were pretreated with 0, 10, 50, 100 or 200 μM cinchonine prior to treatment with 200 μM neomycin. With increasing doses of cinchonine, there was increasing hair cell survival. The negative control represents fish not exposed to neomycin. Data bars are mean hair cell survival from 8 to 10 fish. Error bars represent standard deviation from the mean.

The structures of the nine compounds are listed below.

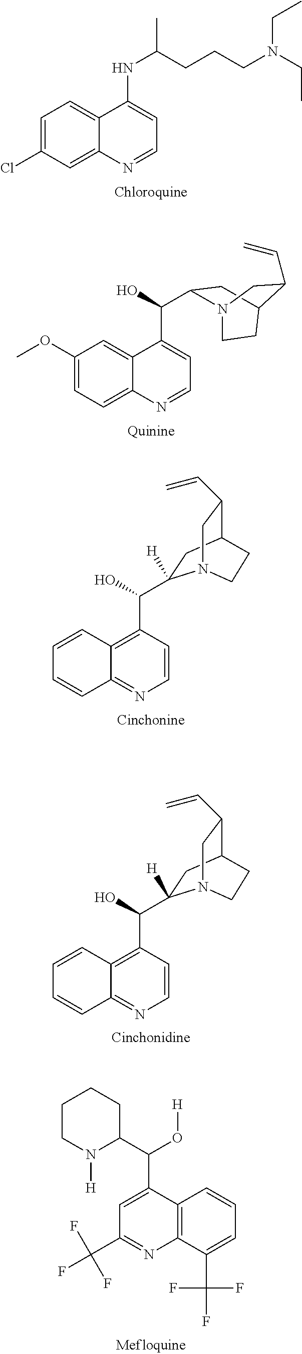

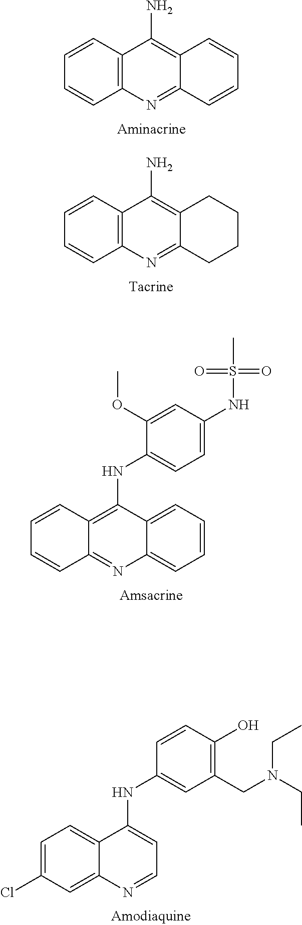

### Example 3

### Protection of Zebrafish Lateral Line Hair Cells is Predictive of Protection of Mammalian Inner Ear Hair Cells

This Example confirms that the observations described in the preceding examples using zebrafish lateral line hair cells provide useful and predictive information about efficacy of protection for hair cells in mammals. In particular, the Example shows that drugs shown to protect hair cells of the zebrafish lateral line from aminoglycoside-induced death are also able to protect rats from aminoglycoside-induced hearing loss.

Rats were injected with 25 mg/kg per day (intraperitoneal) kanamycin for two weeks, a treatment that induces a hearing loss similar to that observed in humans following aminoglycoside treatment. At higher frequencies, the hearing loss observed in rats treated with kanamycin (without protectant) is a threshold shift of 50 dB and at mid-frequencies a hearing loss observed is 30-40 dB threshold shift.

The protectant PROTO1, 2-({[(4-chlorophenyl)amino]carbonyl}amino)-6-ethyl-4,5,6,7-tetrahydrothieno[2,3-c]pyridine-3-carboxamide (identified as F5 in US 2009 0023751-A1), was administered to rats in the experimental group, and was shown to provide significant protection against hearing loss in this animal model. The PROTO1 was administered intraperitoneally at a dose of 25 mg/kg. The PROTO1 was given as a 3.33 mg/mL solution in 1:1:2:16 (DMSO:Cremophor EL:EtOH:PBS).

Thus protectants, exemplified by PROTO1, shown to protect sensory hair cells against ototoxins in the Zebrafish model can also be used to protect sensory hair cells of the mammalian inner ear. The protectants described herein offer a means to attenuate, if not avoid, the hearing loss typically observed with ototoxic medications.

Throughout this application various publications are referenced. The disclosures of these publications in their entireties are hereby incorporated by reference into this application in order to describe more fully the state of the art to which this invention pertains.

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended claims.

What is claimed is:

1. A method of attenuating sensory hair cell death in a subject, the method comprising administering to the subject a sufficient amount of a protective drug selected from the group consisting of: aminophylline (317-34-0), atovaquone (95233-18-4), benzamil (2898-76-2), cefepime (88040-23-7), chloroquine phosphate (50-63-5), fluoxetine HCl (56296-78-7), fluperlapine (67121-76-0), fluspirilene (1841-19), loperamide (34552-83-5), methiothepin maleate (19728-88-2), paroxetine HCl (110429-49-8), phenoxybenzamine HCl (63-92-3), ractopamine (97825-25-7), raloxifene HCl (82640-04-8), sildenafil (139755-83-2), tamoxifen citrate (54965-24-1), ticlopidine HCl (53885-35-1), trequinsin (79855-88-2), trifluperidol 2HCl (749-13-3), toremifene (89778-26-7), quinine, cinchonine, cinchonidine, mefloquine, aminacrine, tacrine, amsacrine, and amodiaquine.

2. A method of reducing ototoxic effects of ototoxic medication in a subject, the method comprising administering to the subject a sufficient amount of a protective drug selected from the group consisting of: aminophylline (317-34-0), atovaquone (95233-18-4), benzamil (2898-76-2), cefepime (88040-23-7), chloroquine phosphate (50-63-5), fluoxetine HCl (56296-78-7), fluperlapine (67121-76-0), fluspirilene (1841-19), loperamide (34552-83-5), methiothepin maleate (19728-88-2), paroxetine HCl (110429-49-8), phenoxybenzamine HCl (63-92-3), ractopamine (97825-25-7), raloxifene HCl (82640-04-8), sildenafil (139755-83-2), tamoxifen citrate (54965-24-1), ticlopidine HCl (53885-35-1), trequinsin (79855-88-2), trifluperidol 2HCl (749-13-3), toremifene (89778-26-7), quinine, cinchonine, cinchonidine, mefloquine, aminacrine, tacrine, amsacrine, and amodiaquine.

3. The method of claim 2, wherein the protective drug is administered prior to administration of the ototoxic medication.

4. The method of claim 3, wherein the protective drug is administered at least about 10 minutes prior to administration of the antibiotic or anti-neoplastic medication.

5. The method of claim 3, wherein the protective drug is administered about 45-75 minutes prior to administration of the ototoxic medication.

6. The method of claim 3, wherein the protective drug is administered daily for 1-7 days.

7. The method of claim 2, wherein the protective drug is administered simultaneously with administration of the ototoxic medication.

8. The method of claim 2, wherein the protective drug is administered after administration of the ototoxic medication.

9. The method of claim 2, wherein the drug is administered orally, intraperitoneally, intramuscularly, intra-aurally, transtympanically or intravenously.

10. The method of claim 2, wherein the ototoxic effects comprise hearing loss.

11. The method of claim 2, wherein the ototoxic effects comprise aminoglycoside entry into hair cells.

12. The method of claim 11, wherein the aminoglycoside is gentamicin or neomycin.

13. The method of claim 2, wherein the protective drug is benzamil, loperamide, ractopamine, raloxifene, paroxetine, phenoxybenzamine, chloroquine, methiothepin, fluoxetine, fluspirilene, tamoxifen, or toremifene.

14. The method of claim 2, wherein the ototoxic medication is neomycin, gentamicin, kanamycin, tobramycin, amikacin, cisplatin or carboplatin.

15. A pharmaceutical composition comprising an ototoxic medication and at least one protective drug, wherein the protective drug is selected from the group consisting of: benzamil, loperamide, ractopamine, raloxifene, paroxetine, phenoxybenzamine, chloroquine, methiothepin, fluoxetine, fluspirilene, tamoxifen, toremifene, quinine, cinchonine, cinchonidine, mefloquine, aminacrine, tacrine, amsacrine, and amodiaquine.

16. The composition of claim 15, wherein the ototoxic medication is neomycin, gentamicin, kanamycin, tobramycin, amikacin, cisplatin or carboplatin.
